(19) United States
(12) Patent Application Publication
(10) Pub. No.: US 2003/0096834 A1
(43) Pub. Date: May 22, 2003

(54) Pharmaceutical compositions

(76) Inventors: Richard John Jenkins, Durham, NC (US); David Hugh Richards, Ware, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/240,868

(22) PCT Filed: Apr. 5, 2001
(86) PCT No.: PCT/EP01/03889

(30) Foreign Application Priority Data
Apr. 7, 2000 (GB) 0008485.5

(51) Int. Cl. A61L 9/04; A61K 31/4745; A61K 31/137

(52) U.S. Cl. 514/291; 424/46; 514/649

(57) ABSTRACT

The present invention is concerned with pharmaceutical formulations comprising a combination of salmeterol and ipratropium and the use of such formulations in medicine, particularly in the prophylaxis and treatment of respiratory diseases.

The present invention is concerned with compositions containing a combination of salmeterol and ipratropium and the use of such compositions in medicine, particularly in the prophylaxis and treatment of respiratory diseases.

GB 2 140 800 describes phenethanolamine compounds which are β₂-adrenoreceptor agonists including 4-hydroxy-α¹-[[[6-(4-phenylbutoxy)hexyl]-amino]methyl]-1,3-benzenedimethanol 1-hydroxy-2-naphthalenecarboxylate (salmeterol xinafoate) which is now used clinically in the treatment of bronchial asthma and related disorders.

U.S. Pat. No. 3,505,337 and U.S. Pat. No. 3,681,500 describe ipratropium and its salts, such (endo,syn)-(+/−)-3-(3-hydroxy-1-oxo-2-phenylpropoxy)-8-methyl-8-(1-methylethyl)-8-azoniabicyclo[3.2.1]octane bromide (ipratropium bromide) and pharmaceutical formulations thereof. Ipratropium bromide is an anticholinergic agent, which is now used clinically in the treatment of bronchial asthma and related disorders.

Although salmeterol xinafoate and ipratropium bromide are effective bronchodilators, the maximum duration of action is 12 hours and 6 hours respectively, hence twice daily or four times daily dosing is often required. There is therefore a clinical need for bronchodilators having potent and selective action and having an advantageous profile of action

According to the present invention, there is provided a pharmaceutical formulation comprising salmeterol or a pharmaceutically acceptable salt, solvate, or physiologically functional derivative thereof and ipratropium or a pharmaceutically acceptable salt, solvate, or physiologically functional derivative thereof, and a pharmaceutically acceptable carrier or excipient, and optionally one or more other therapeutic ingredients. According to a preferred aspect of the present invention, there is provided a pharmaceutical formulation comprising salmeterol xinafoate and ipratropium bromide, and a pharmaceutically acceptable carrier or excipient, and optionally one or more other therapeutic ingredients. In the most preferred aspect, the above pharmaceutical formulations are suitable for administration by inhalation.

It is to be understood that the present invention covers all combinations of particular and preferred aspects of the invention described herein.

As would be appreciated by the skilled person, salmeterol includes an asymmetric centre and ipratropium includes three asymmetric centres. The present invention includes both (S) and (R) enantiomers of salmeterol either in substantially pure form or admixed in any proportions, as well as each isomer of ipratropium either in substantially pure form or admixed in any proportions. The enantiomers of salmeterol have been described previously, for example, in EP0422889 and WO 99/13867. Various isomers of ipratropium are described in DE 4140861 and WO 97/05136.

By the term “physiologically functional derivative” is meant a chemical derivative of salmeterol or ipratropium having the same physiological function as the free compound, for example, by being convertible in the body thereto. According to the present invention, examples of physiologically functional derivatives include esters.

Suitable salts according to the invention include those formed with both organic and inorganic acids. Pharmaceutically acceptable acid addition salts include but are not limited to those formed from hydrochloric, hydrobromic, sulphuric, citric, tartaric, phosphoric, lactic, pyruvic, acetic, trifluoroacetic, succinic, oxalic, fumaric, maleic, oxaloacetic, methanesulphonic, ethanesulphonic, p-toluenesulphonic, benzenesulphonic, isethionic, and naphthalenecarboxylic, such as 1-hydroxy-2-naphthalenecarboxylic acids.

Pharmaceutically acceptable esters of salmeterol or ipratropium may have a hydroxyl group converted to a C₁₋₆ alkyl, aryl, aryl C₁₋₆ alkyl, or amino acid ester.

As mentioned above, both salmeterol and ipratropium and their pharmaceutically acceptable salts, solvates, and physiologically functional derivatives have been described for use in the treatment of respiratory diseases. Therefore, formulations of salmeterol and ipratropium and their pharmaceutically acceptable salts, solvates, and physiologically functional derivatives have use in the prophylaxis and treatment of clinical conditions for which a selective β₂-adrenoreceptor agonist and/or an anticholinergic agent is indicated. Such conditions include diseases associated with reversible airways obstruction such as asthma, chronic obstructive pulmonary diseases (COPD) (e.g. chronic and wheezy bronchitis, emphysema), respiratory tract infection and upper respiratory tract disease (e.g. rhinitis, such as allergic and seasonal rhinitis).

Accordingly, the present invention provides a method for the prophylaxis or treatment of a clinical condition in a mammal, such as a human, for which a selective β₂-adrenoreceptor agonist and/or anticholinergic agent is indicated, which comprises administration of a therapeutically effective amount of a pharmaceutical formulation comprising salmeterol or a pharmaceutically acceptable salt, solvate, or physiologically functional derivative thereof and ipratropium or a pharmaceutically acceptable salt, solvate, or physiologically functional derivative thereof, and a pharmaceutically acceptable carrier or excipient. In a preferred aspect, there is provided such a method which comprises administration of a therapeutically effective amount of a pharmaceutical formulation comprising salmeterol xinafoate and ipratropium bromide, and a pharmaceutically acceptable carrier or excipient. In particular, the present invention provides such a method for the prophylaxis or treatment of a disease associated with reversible airways obstruction such as asthma, chronic obstructive pulmonary disease (COPD), respiratory tract infection or upper respiratory tract disease.

The amount of salmeterol and ipratropium, or a pharmaceutically acceptable salt, solvate or physiologically functional derivative thereof which is required to achieve a therapeutic effect will, of course, vary with the particular compound, the route of administration, the subject under treatment, and the particular disorder or disease being treated. As a monotherapy, salmeterol xinafoate is generally administered to adult humans by aerosol inhalation at a dose of 50 mcg or 100 mcg twice daily. As a monotherapy, ipratropium bromide is generally administered to adult humans by inhalation at a dose of from 20 mcg to 80 mcg three or four times daily.

Hereinafter, the term “active ingredients” means salmeterol or a pharmaceutically acceptable salt, solvate, or physiologically functional derivative thereof, preferably salmeterol xinafoate, and ipratropium, or a pharmaceutically acceptable salt, solvate, or physiologically functional derivative thereof, preferably ipratropium bromide.

Suitably, the pharmaceutical formulations which are suitable for inhalation according to the invention comprise the active ingredients in amounts such that each actuation provides therapeutically effective dose, for example, a dose of salmeterol of 10 mcg to 150 mcg, preferably 50 mcg and a dose of ipratropium bromide of 10 mcg to 400 mcg, preferably 50 mcg to 320 mcg, more preferably, 80 mcg to 160 mcg. The pharmaceutical formulations according to the invention may further include other therapeutic agents for example anti-inflammatory agents such as corticosteroids or other β₂-adrenoreceptor agonists (such as salbutamol, formoterol, fenoterol or terbutaline and salts thereof).

Suitably, the pharmaceutical formulations which are suitable for inhalation according to the invention provide therapeutically effective doses that permit the establishment of a twice daily (bis in diem—b.i.d) dosing regimen.

The formulations include those suitable for oral, parenteral (including subcutaneous, intradermal, intramuscular, intravenous and intraarticular), intranasal, inhalation (including fine particle dusts or mists which may be generated by means of various types of metered dose pressurised aerosols, nebulisers or insufflators), rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing the active ingredients into association with the carrier which constitutes one or more accessory ingredients. In general the formulations are prepared by uniformly and intimately bringing into association the active ingredients with liquid carriers or finely divided solid carriers or both and then, if necessary, shaping the product into the desired formulation.

Formulations for inhalation include powder compositions which will preferably contain lactose, and spray compositions which may be formulated, for example, as aqueous solutions or suspensions or as aerosols delivered from pressurised packs, with the use of a suitable propellant, e.g. dichlorodifluoromethane, trichlorofluoromethane, dichlorotetrafluoroethane, 1,1,1,2,3,3,3-heptafluoropropane, 1,1,1,2-tetrafluoroethane, carbon dioxide or other suitable gas. Suitable aerosol formulations include those described in EP 0372777 and WO93/11743. Suitable aerosol formulations may include excipients such as surfactants and/or cosolvents such as ethanol. For suspension aerosols, the active ingredients should be micronised so as to permit inhalation of substantially all of the active ingredients into the lungs upon administration of the aerosol formulation, thus the active ingredients will have a particle size of less than 100 microns, desirably less than 20 microns, and preferably in the range 1 to 10 microns, for example, 1 to 5 microns.

Preferred compositions for aerosol delivery consist essentially of particulate active ingredients, and 1,1,1,2-tetrafluoroethane, 1,1,1,2,3,3,3-heptafluorpropane or mixtures thereof as propellant. Especially preferred compositions for aerosol delivery consist of particulate active ingredients, and 1,1,1,2-tetrafluoroethane, 1,1,1,2,3,3,3-heptafluorpropane or mixtures thereof as propellant.

Compositions according to the present invention for aerosol delivery may be filled into canisters suitable for delivering pharmaceutical aerosol formulations. Canisters generally comprise a container capable of withstanding the vapour pressure of the propellant, such as plastic or plastic-coated glass bottle or preferably a metal can, for example an aluminium can which may optionally be anodised, lacquer-coated and/or plastic-coated, which container is closed with a metering valve. It may be preferred that canisters be coated with a fluorocarbon polymer as described in WO 96/32150, for example, a co-polymer of polyethersulphone (PES) and polytetrafluoroethylene (PTFE). Another polymer for coating that may be contemplated is FEP (fluorinated ethylene propylene). The metering valves are designed to deliver a metered amount of the formulation per actuation and incorporate a gasket to prevent leakage of propellant through the valve. The gasket may comprise any suitable elastomeric material such as for example low density polyethylene, chlorobutyl, black and white butadiene-acrylonitrile rubbers, butyl rubber and neoprene. Thermoplastic elastomer valves as described in WO92/11190 and valves containing EPDM rubber as described in WO95/02650 are especially suitable. Suitable valves are commercially available from manufacturers well known in the aerosol industry, for example, from Valois, France (eg. DF10, DF30, DF60), Bespak pic, UK (eg. BK300, BK356, BK357) and 3M-Neotechnic Ltd, UK (eg. Spraymiser™). The DF31 valve of Valois, France is also suitable.

Valve seals, especially the gasket seal and also the seals around the metering chamber, will preferably be manufactured of a material which is inert to and resists extraction into the contents of the formulation, especially when the contents include ethanol.

Valve materials, especially the material of manufacture of the metering chamber, will preferably be manufactured of a material which is inert to and resists distortion by contents of the formulation, especially when the contents include ethanol. Particularly suitable materials for use in manufacture of the metering chamber include polyesters eg polybutyleneterephthalate (PBT) and acetals, especially PBT.

Materials of manufacture of the metering chamber and/or the valve stem may desirably be fluorinated, partially fluorinated or impregnated with fluorine containing substances in order to resist drug deposition.

Valves which are entirely or substantially composed of metal components (eg Spraymiser, 3M-Neotechnic) are especially preferred for use according to the invention.

Intranasal sprays may be formulated with aqueous or non-aqueous vehicles with the addition of agents such as thickening agents, buffer salts or acid or alkali to adjust the pH, isotonicity adjusting agents or anti-oxidants.

Capsules and cartridges or for example gelatin, or blisters of for example laminated aluminium foil, for use in an inhaler or insuflator may be formulated containing a powder mix of the active ingredients and a suitable powder base such as lactose or starch, preferably lactose. In this aspect, the active ingredients are suitably micronised so as to permit inhalation of substantially all of the active ingredients into the lungs upon administration of the dry powder formulation, thus the active ingredients will have a particle size of less than 100 microns, desirably less than 20 microns, and preferably in the range 1 to 10 microns.

Solutions for inhalation by nebulation may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Preferred unit dosage formulations are those containing a pharmaceutically effective dose, as hereinbefore recited, or an appropriate fraction thereof, of the active ingredient. Thus, in the case of formulations designed for delivery by metered dose pressurised aerosols, one actuation of the aerosol may deliver half of the therapeutically effective amount such that two actuations are necessary to deliver the therapeutically effective dose.

It should be understood that in addition to the ingredients particularly mentioned above, the formulations of this invention may include other agents conventional in the art having regard to the type of formulation in question. Furthermore, the claimed formulations include bioequivalents as defined by the US Food and Drugs Agency.

For a better understanding of the invention, the following Examples are given by way of illustration.

### EXAMPLES

A: Metered Dose Inhalers

### Example 1

### 25/40 Salmeterol/Ipratropium

| | Per Inhaler % w/w | Per actuation |
|---|---|---|
| Salmeterol Xinafoate | 0.05 | 36.3 microgram |
| Ipratropium Bromide | 0.05 | 40 microgram |
| 1,1,1,2-Tetrafluoroethane | to 100 | to 75.0 mg |

The micronised active ingredients are weighed into an aluminium can, 1,1,1,2-tetrafluoroethane is then added from a vacuum flask and a metering valve with a 63 μl metering chamber is crimped into place.

Similar methods may be used for the formulations of Examples 2 to 8:

### Example 2

### 25/60 Salmeterol/Ipratropium

| | Per Inhaler % w/w | Per actuation |
|---|---|---|
| Salmeterol Xinafoate | 0.05 | 36.3 microgram |
| Ipratropium Bromide | 0.08 | 60 microgram |
| 1,1,1,2-Tetrafluoroethane | to 100 | to 75.0 mg |

### Example 3

### 25/80 Salmeterol/Ipratropium

| | Per Inhaler % w/w | Per actuation |
|---|---|---|
| Salmeterol Xinafoate | 0.05 | 36.3 microgram |
| Ipratropium Bromide | 0.11 | 80 microgram |
| 1,1,1,2-Tetrafluoroethane | to 100 | to 75.0 mg |

### Example 4

### 25/160 Salmeterol/Ipratropium

| | Per Inhaler % w/w | Per actuation |
|---|---|---|
| Salmeterol Xinafoate | 0.05 | 36.3 microgram |
| Ipratropium Bromide | 0.21 | 160 microgram |
| 1,1,1,2-Tetrafluoroethane | to 100 | to 75.0 mg |

In Examples 5 to 8, a metering valve with a 50 μl metering chamber is used.

### Example 5

### 25/40 Salmeterol/Ipratropium

| | Per Inhaler % w/w | Per actuation |
|---|---|---|
| Salmeterol Xinafoate | 0.06 | 36.3 microgram |
| Ipratropium Bromide | 0.07 | 40 microgram |
| 1,1,1,2-Tetrafluoroethane | to 100 | to 60.0 mg |

### Example 6

### 25/60 Salmeterol/Ipratropium

| | Per Inhaler % w/w | Per actuation |
|---|---|---|
| Salmeterol Xinafoate | 0.06 | 36.3 microgram |
| Ipratropium Bromide | 0.10 | 60 microgram |
| 1,1,1,2-Tetrafluoroethane | to 100 | to 60.0 mg |

### Example 7

### 25/80 Salmeterol/Ipratropium

| | Per Inhaler % w/w | Per actuation |
|---|---|---|
| Salmeterol Xinafoate | 0.06 | 36.3 microgram |
| Ipratropium Bromide | 0.13 | 80 microgram |
| 1,1,1,2-Tetrafluoroethane | to 100 | to 60.0 mg |

### Example 8

### 25/160 Salmeterol/Ipratropium

| | Per Inhaler % w/w | Per actuation |
|---|---|---|
| Salmeterol Xinafoate | 0.06 | 36.3 microgram |
| Ipratropium Bromide | 0.27 | 160 microgram |
| 1,1,1,2-Tetrafluoroethane | to 100 | to 60.0 mg |

### Example 9

### 25/80 Salmeterol/Ipratropium

| | Per Inhaler % w/w | Per actuation |
|---|---|---|
| Salmeterol Xinafoate | 0.05 | 36.3 microgram |
| Ipratropium Bromide | 0.11 | 80 microgram |
| 1,1,1,2-Tetrafluoroethane | to 100 | to 75.0 mg |

The micronised active ingredients were weighed into an aluminium can coated internally with a PTFE/PES polymer blend as described in WO96/32150, 1,1,1,2-tetrafluoroethane was then added from a vacuum flask and a Valois DF60 metering valve (metering chamber volume 63 μl) was crimped into place.

In an alternative process, the formulation of Example 9 was also made up by weighing the micronised active ingredients into a pressurised vessel and adding a portion of the 1,1,1,2-tetrafluoroethane to form a suspension. Aliquots of the suspension were filled, through the valve, into a number of aluminium cans coated internally with a PTFE/PES polymer blend as described in WO96132150 and closed with Valois DF60 metering valves. Further 1,1,1,2-tetrafluoroethane (to 100% w/w) was then added to each can through the valve.

B: Dry Powder Inhalers

### Example 10

### 50/80 Salmeterol/Ipratropium

| | Per cartridge or blister |
|---|---|
| Salmeterol Xinafoate | 72.6 microgram |
| Ipratropium Bromide | 80 microgram |
| Lactose Ph. Eur. | to 12.5 mg |
| | or to 25.0 mg |

The active ingredients are micronised and bulk blended with the lactose in the proportions given above. The blend is filled into hard gelatin capsules or cartridges or in specifically constructed double foil blister packs to be administered by an inhaler such as a Rotahaler, Diskhaler, or Diskus inhaler (each of these being a Trademark of Glaxo Group Limited).

Similar methods may be used for the formulations of Examples 11 to 13:

### Example 11

### 50/120 Salmeterol/Ipratropium

| | Per cartridge or blister |
|---|---|
| Salmeterol Xinafoate | 72.6 microgram |
| Ipratropium Bromide | 120 microgram |
| Lactose Ph. Eur. | to 12.5 mg |
| | or to 25.0 mg |

### Example 12

### 50/160 Salmeterol/Ipratropium

| | Per cartridge or blister |
|---|---|
| Salmeterol Xinafoate | 72.6 microgram |
| Ipratropium Bromide | 160 microgram |
| Lactose Ph. Eur. | to 12.5 mg |
| | or to 25.0 mg |

### Example 13

### 50/320 Salmeterol/Ipratropium

| | Per cartridge or blister |
|---|---|
| Salmeterol Xinafoate | 72.6 microgram |
| Ipratropium Bromide | 320 microgram |
| Lactose Ph. Eur. | to 12.5 mg |
| | or to 25.0 mg |

### Example 14

### 50/80 Salmeterol/Ipratropium

| | Per cartridge or blister |
|---|---|
| Salmeterol Xinafoate | 72.5 microgram |
| Ipratropium Bromide | 80 microgram |
| Lactose Ph. Eur. | to 12.5 mg |

The active ingredients were micronised and bulk blended with the lactose in the proportions given above (total blend size 4 kg). The blend was filled into specifically constructed double foil blister packs to be administered by a Diskus inhaler (Trademark of Glaxo Group Limited).

A similar method was used for the formulation of Example 15. The total blend size was 4 kg.

### Example 15

### 50/160 Salmeterol/Ipratropium

| | Per cartridge or blister |
|---|---|
| Salmeterol Xinafoate | 72.5 microgram |
| Ipratropium Bromide | 160 microgram |
| Lactose Ph. Eur. | to 12.5 mg |

1. A pharmaceutical formulation comprising salmeterol or a pharmaceutically acceptable salt, solvate, or physiologically functional derivative thereof and ipratropium or a pharmaceutically acceptable salt, solvate, or physiologically functional derivative thereof, and a pharmaceutically acceptable carrier or excipient, and optionally one or more other therapeutic ingredients.

2. A pharmaceutical formulation comprising salmeterol xinafoate and ipratropium bromide, and a pharmaceutically acceptable carrier or excipient, and optionally one or more other therapeutic ingredients.

3. A pharmaceutical formulation according to claim 1 or 2 which is suitable for administration by inhalation.

4. A pharmaceutical formulation according to any of claims 1 to 3 which is a dry powder.

5. A pharmaceutical formulation according to any of claims 1 to 4 wherein the pharmaceutically acceptable carrier or excipient is lactose.

6. A pharmaceutical formulation according to any of claims 1 to 3 which is an aerosol formulation.

7. A pharmaceutical formulation according to any of claims 1 to 3 wherein the pharmaceutically acceptable carrier or excipient comprises a propellant.

8. A pharmaceutical formulation according to claim 6 wherein the propellant comprises 1,1,1,2-tetrafluoroethane and/or 1,1,1,2,3,3,3-heptafluorpropane.

9. A pharmaceutical formulation consisting essentially of particulate salmeterol or a pharmaceutically acceptable salt, solvate, or physiologically functional derivative thereof and particulate ipratropium or a pharmaceutically acceptable salt, solvate, or physiologically functional derivative thereof, and optionally one or more other therapeutic ingredients, and 1,1,1,2-tetrafluoroethane, 1,1,1,2,3,3,3-heptafluorpropane or mixtures thereof as propellant.

10. A pharmaceutical formulation consisting essentially of particulate salmeterol xinafoate and particulate ipratropium bromide, and optionally one or more other therapeutic ingredients, and 1,1,1,2-tetrafluoroethane, 1,1,1,2,3,3,3-heptafluorpropane or mixtures thereof as propellant.

11. A method for the prophylaxis or treatment of a clinical condition in a mammal, such as a human, for which a selective β₂-adrenoreceptor agonist and/or anticholinergic agent is indicated, which comprises administration of a therapeutically effective amount of a pharmaceutical formulation according to any one of claims 1 to 10.

12. A method according to claim 11 wherein the clinical condition is a disease associated with reversible airways obstruction such as asthma, chronic obstructive pulmonary disease (COPD), respiratory tract infection or upper respiratory tract disease.

13. A method according to claim 11 wherein the clinical condition is chronic obstructive pulmonary disease (COPD).
